(19) United States
(12) Patent Application Publication
(10) Pub. No.: US 2020/0402618 A1
(43) Pub. Date: Dec. 24, 2020

(54) GENE SEQUENCING QUALITY LINE DATA COMPRESSION PRE-PROCESSING AND DECOMPRESSION AND RESTORATION METHODS, AND SYSTEM

(71) Applicant: GENETALKS BIO-TECH (CHANGSHA) CO., LTD., Hunan (CN)

(72) Inventors: Yanhuang JIANG, Hunan (CN); Zhuo SONG, Hunan (CN); Gen LI, Hunan (CN); Qiangli ZHAO, Hunan (CN); Bolun FENG, Hunan (CN); Hongwei TANG, Hunan (CN); Xiali XU, Hunan (CN); Haibo MAO, Hunan (CN)

(73) Assignee: GENETALKS BIO-TECH (CHANGSHA) CO., LTD., Hunan (CN)

(21) Appl. No.: 16/969,197

(22) PCT Filed: Apr. 12, 2019
(86) PCT No.: PCT/CN2019/082466

§ 371 Date: Aug. 12, 2020

(30) Foreign Application Priority Data
Apr. 27, 2018 (CN) 201810392727.7

(51) Int. Cl. G16B 50/50; G16B 30/00; G16B 50/30; H03M 7/30

(52) U.S. Cl. CPC G16B 50/50; H03M 7/30; G16B 50/30; G16B 30/00

(57) ABSTRACT

This invention relates to a gene sequencing quality line data compression pre-processing and decompression and restoration method, and a system, wherein the basic principle of the gene sequencing quality line data compression pre-processing and decompression and restoration is to extract several columns from an inputted quality line document or data block to act as index columns, and then perform rearrangement on all quality line data, all quality lines having a same index column being one group and being arranged together according to their relative positions in the original data block. Since quality line data having a same index column is usually more similar, the data reorganization means can arrange similar gene sequencing data together, so as to increase local similarity of the data.

### BACKGROUND

### Technical Field

The present invention relates to gene sequencing quality line data compression pre-processing and decompression technology, in particular to gene sequencing quality line data compression pre-processing and decompression and restoration methods, and a system.

### Description of Related Art

Gene detection is a technology capable of detecting DNA through blood, other body fluids or cells, and a method capable of detecting DNA molecule information in the cells of a detected person and analyzing whether gene types, defects and expression functions contained therein are normal, through which people can know their gene information, determine the disease causes or predict the body's risk for a certain disease. Gene detection can be used for disease diagnosis and disease risk prediction. As the gene sequencing technology upgrades continuously, the sequencing throughput is getting higher and higher, and meanwhile the sequencing cost is plummeting. Hence, a high-throughput sequencing technology has been gradually used in scientific research, medical treatment and other fields gradually. In the meantime, as people's living standards improve, the number of people using the gene detection technology to diagnose and predict diseases is also increasing. This leads to a huge increase in the amount of sequencing data generated by the gene detection technology. Storage and transportation of massive gene sequencing data have been an important technical problem encountered in the gene detection application. A lossless compression algorithm with a high compression ratio is an important technical approach to solve this difficulty. Quality line data compression in the gene sequencing result is also a difficulty in the gene sequencing data compression.

A current compression processing strategy for the quality line data in the gene sequencing is to obtain a good compression efficiency by performing compression pre-processing (such as change of a data order), and then using a classical compression algorithm. The most common method is to: pre-process using a BWT algorithm, and then compress by virtue of arithmetic coding. Compression pre-processing aims to put the same or similar data together as much as possible, and then use the compression algorithm to improve the compression efficiency.

As the most common compression pre-processing method, Burrows-Wheeler Transform (BWT) is mainly based on the following ideas: circularly shifting an original character string (S) with length of (N) rightwards in turn to obtain (N) character strings, and then sorting the (N) character strings in lexicographic order. The original character string (S) can be restored by only saving a character string (L) consisting of end characters of (N) character strings sorted and the positions of the original characters (S) on the (N) character strings. The BWT algorithm mainly includes the following critical steps:

(1) obtaining the character strings shifting rightwards circularly: making the length of the original character string (S) as (N), circularly shifting the same rightwards, that is, the (N) character strings can be obtained by repeatedly moving one bit rightwards in turn till a last bit is moved to a first bit;

(2) sorting the character strings shifted: sorting the (N) character strings obtained by circularly shifting rightwards in lexicographic order, to obtain a character matrix (M);

(3) obtaining the pre-processed data: obtaining the character string (L) consisting of the last column of characters thereof according to the character matrix (X), namely: L[k]=M[k,N−1](0≤k≤N−1), a k^{th} character of the (L) is the last character of the k^{th} line of the matrix (M). The original character string (S) is located at the I^{th} line of the (M), namely: M[I,j]=S[j](0≤j≤N−1), a pre-processing result (L, I) is exported.

The BWT algorithm needs to restore the original character string (S) based on (L, I) during the decompression. The specific processing procedures are as follows:

(1) calculating a character string (F) consisting of a first column of characters of the matrix M in pre-processing: the characters in (L) are sorted in lexicographic order to obtain a character string (F) due to the fact that the matrix (M) is sorted in lexicographic order;

(2) determining a correlation between the characters in (L) and (F): if a matrix (M′) is supposed that the matrix M moves one bit rightwards circularly, it can be seen that a first column of (M′) is (L); on account that a second column of M′ is the same as a first column of the matrix (M), which is a result of sorting in lexicographic order, it can be seen that the occurrence sequences of the same letters in (L) and (F) are the same, and thus L[j]=F[T[j]], the correlation (T) between the characters in (L) and (F) can be established;

(3) obtaining the original character string (S): (F[i]) and (L[i]) are a first character and a last character of the i^{th} line in (M) respectively due to the fact that the character strings in the matrix (M) are both obtained by shifting the original character string (S) rightwards circularly, and thus (L[i]) is located in front of (F[i]) all the time in rightwards shifting circularly. According to a relation vector (T) between (L) and (F), each character in (S) can be calculated sequentially from back to front by the following method: S[N−1−i]=L[Ti[I]]0≤i≤N−1), where T0[x]=x, Ti+1[x]=T[Ti[x]]. Thus, the original character string (S) is obtained.

BWT is an efficient compression pre-processing method, which adjusts the sequence of the characters in the character string to be compressed by means of shifting rightwards circularly, so that the same or similar characters can be arranged together to improve the subsequent compression efficiency. However, the BWT algorithm has the following two defects: (1) High extra overhead: extra storage overhead is introduced at the pre-processing stage due to the fact that the BWT algorithm needs to save location information (I) of the original character string (S) in the matrix (M). This extra overhead may result in that the compression efficiency cannot be improved by the pre-processed result. (2) Small pre-processing window: The BWT algorithm only adjusts the sequence of the characters in the character string, the pre-processing window thereof is only the character string with the fixed length; the small pre-processing window is small and does not consider reordering the data blocks from the perspective of files or big blocks.

In a context of massive data, the BWT algorithm is limited to improve the data similarity in the big data blocks due to small pre-processing window. Besides, the compression efficiency is limited to be further improved by the extra overhead during the pre-processing thereof.

### SUMMARY

The technical problem to be solved by the present invention is to, with respect to the above problems in the prior art, provide gene sequencing quality line data compression pre-processing and decompression and restoration methods, and a system. The present invention does not introduce the additional storage overhead, and uses only small computational overhead to implement data rearrangement within the big data windows, so as to improve compression efficiency. The present invention is suitable for performing compression pre-processing on quality line data during gene sequencing, wherein the data block is larger, the advantage is more significant.

For the purpose of solving the above technical problem, the technical solution applied by the present invention is as follows:

The present invention provides a gene sequencing quality line data compression pre-processing method, including:

1) reading an original data block (Data) of the quality line data and determining an index column numbers (Index_No) thereof;

2) establishing the index information table (IIT) according to the index columns of the original data block (Data);

3) according to the index information table (IIT), regrouping quality lines in the original data block (Data) according to the index column information, and deleting index column portion data to obtain grouped data (Grouped_Data);

4) extracting index column data (Index_Data) of the original data block (Data), and exporting the index column numbers (Index_No), index column data (Index_Data) of the original data block (Data) and data (Grouped_Data) regrouped as the compression pre-processing results.

Preferably, step 2) includes the following detailed steps:

2.1) initializing the number of entries of the index information table (IIT) to be 0, and including serial numbers, index column information (Index), and variables (num, start and temp) in entries of the index information table (IIT) structurally, wherein the variable (num) is the number of quality lines having the corresponding index column information; the variable (start) indicates initial locations of the quality lines having the index column information after grouped; the variable (temp) is the number of quality lines having the corresponding index column information in regrouping.

2.2) initializing the current quality line number (i) of the original data block (Data) to be 0;

2.3) sequentially scanning the current quality line (Data[i]) in the original data block (Data), and jumping to execute step 2.6) if reaching the end of the original data block(Data); otherwise, taking out the index column information (Index) of the current quality line (Data[i]), wherein (Data[i]) refers to the contents of the current quality line (i) in the original data block (Data); adding 1 to the current quality line number (i);

2.4) searching all entries in the index information table (IIT), adding 1 to the variable (num) of the entry (j) if the index column information of a certain entry (j) of the index information table (IIT) is equal to the index column information (Index) of the current quality line (Data[i]), jumping to execute step 2.3); otherwise, jumping to execute step 2.5);

2.5) establishing a new entry (k) in the index information table (IIT), setting index column information (IIT[k].Index) of an entry (k) to be equal to index column information (Index) of the current quality line (Data[i]), and the variable (num) of the entry (k) to be equal to 1, and adding 1 to a serial number (k); jumping to execute step 2.3);

2.6) initializing the current entry (j) of the index information table (IIT) to be 0;

2.7) sequentially scanning the entries of the index information table (IIT), setting corresponding grouping start positions for all index column information, and ending this step and jumping to execute step 3) if reaching the end of the index information table (IIT); otherwise, with respect to the entry (j) scanned currently in the index information table (IIT), setting the value of the variable (start) of the entry (j) to be 0 and the value of variable (temp) to be 0 if the serial number (j) of the entry is 0, and adding 1 to the current entry number (j); jumping to continue with step 2.7); otherwise setting the value of the variable (start) of the entry (j) to be the sum of the variable (start) and the variable (num) of the last entry (j−1) and the variable (temp) of the entry (j) to be 0, adding 1 to the current entry number, and jumping to continue with step 2.7).

Preferably, step 3) includes the following detailed steps:

3.1) allocating a space for the regrouped data (Grouped_Data), wherein the number of lines thereof is the same as that of the original data block (Data);

3.2) initializing the value of the current quality line number (i) of the original data block (Data) to be 0;

3.3) scanning the current quality line of the original data block (Data), wherein the data of the current quality line is Data[i], and (i) is the current quality line number; taking out the index column information (Index) of the current quality line Data[i];

3.4) searching the entry (j), the index information of which is the same as Index, in the index information table (IIT);

3.5) inserting the quality line data, the index column information of which is deleted, into the regrouped data (Grouped_Data), wherein a value of an insertion position (k) is the sum of the variables (start and temp) of the entry (j); adding 1 to the variable (temp) value of the entry (j);

3.6) adding 1 to the line number (i), judging whether the line number (i) is more than the total line number of the original data block (Data), and jumping to execute step 3.3) if the total line number of the original data block (Data) is not exceeded; otherwise, jumping to execute step 4).

The present invention further provides a gene sequencing quality line data decompression and restoration method, including:

S1) reading decompressed index column data (Index_Data), regrouped data (Grouped_Data) and index column numbers (Index_No), determining the quality line number of the original data block (Data) and character data of each line based on the regrouped data (Grouped_Data) and index column number information (Index_No), and allocating the space for the storage of the original data block (Data);

S2) according to the index column numbers (Index_No), respectively assigning each column of data among the index column data (Index_Data) to the corresponding column, the number of which is recorded by Index_No, in the original data block (Data);

S3) establishing the index information table (IIT) according to the index column data (Index_Data);

S4) sequentially scanning each line of data among the regrouped data (Grouped_Data) according to the index information table (IIT), determining the position of the line in the original data block according to the index information table (IIT) and the index column data (Index_Data), and writing the same into the corresponding quality line of the original data block (Data);

S5) exporting the original data block (Data).

Preferably, step S3) includes the following detailed steps:

S3.1) initializing the value of the entry number (k) of the index information table (IIT) to be 0, and including serial numbers, index column information (Index), and variables (num, start and temp) in the entries of the index information table (IIT) structurally, wherein the variable (num) is the number of quality lines having the corresponding index column information; the variable (start) indicates the initial locations of the quality lines having the index column information after grouping; the variable (temp) is the number of quality lines having the corresponding index column information in data restoration;

S3.2) initializing the value of the current line number (i) of the index column data (Index_Data) to be 0;

S3.3) sequentially scanning the index column data (Index_Data), and jumping to execute step S3.6) if reaching the end of the index column data (Index_Data); otherwise, taking out the current index column information (Index_Data[i]) corresponding to the current line in the index column data (Index_Data);

S3.4) searching all entries in the index information table (IIT), adding 1 to the variable (num) of the entry (j) if the index column information (Index) of the entry (j) is the same as the current index column information (Index_Data[i]), and jumping to execute step S3.3); otherwise, jumping to execute step 3.5);

S3.5) establishing a new entry (k) for the index information table (IIT), wherein the index column information (Index) of the entry (k) is equal to the current index column information (Index_Data[i]), and the variable (num) is equal to 1; adding 1 to the entry number (k), and jumping to execute step S3.3);

S3.6) initializing the current entry (j) of the index information table (IIT) to be 0;

S3.7) sequentially scanning the index information table (IIT), and setting the corresponding grouping start position of the current index column information; in case of reaching the end of the index information table (IIT), jumping to step S4); otherwise, with respect to the entry (j) in the index information table (IIT): if the serial number (j) of the entry (j) is 0, setting the variables (start and temp) to be 0, adding 1 to the serial number (j), and jumping to continue with step S3.7); otherwise, setting the variable (start) of the entry (j) to be the sum of the variables (start and num) of the last entry (j−1), wherein the variable (temp) of the entry (j) is 0; adding 1 to the serial number (j); jumping to continue with step S3.7).

Preferably, step S4) includes the following detailed steps:

S4.1) initializing the value of the current line number (k) of the regrouped data (Grouped_Data) to be 0;

S4.2) obtaining the index column information of the regrouped data (Grouped_Data[k]): if reaching the end of regrouped data (Grouped_Data), jumping to execute step S5); otherwise, scanning the index information table (IIT) to find out the entry (j) of the index information table (IIT) to make it conform to that: the value of the line number (k) is more than or equal to the value of the variable (start) of the entry (j), and less than or equal to the sum of the values of the variable (start) of the entry (j) and the variable (num) thereof, wherein the index column information corresponding to the data (Grouped_Data[k]) of the current line in the regrouped data (Grouped_Data) is the index column information (Index) of the entry (j);

S4.3) combining the data (Grouped_Data[k]) of the current line in the regrouped data (Grouped_Data) and the index column information (Index) of the entry (j) to generate a complete quality line (Temp_Read);

S4.4) obtaining an occurrence order (r) in the quality line having the same index column information of the complete quality line (Temp_Read) in the original data block (Data), wherein the value of the occurrence order (r) is a differential value between the current line number (k) and the variable (start) of the entry (j);

S4.5) sequentially scanning the index column data (Index_Data) to find out the r^{th} index column information to be an entry (t) of the index column information (Index) of the entry (j) in the index information table (IIT), so as to determine the line number (t) of the complete quality line (Temp_Read) in the original data block;

S4.6) writing the complete quality line (Temp_Read) to the line number (t) of the original data block (Data);

S4.7) adding 1 to the current line number (k) of the regrouped data (Grouped_Data);

S4.8) judging whether the current line number (k) is more than the maximum line number of the regrouped data (Grouped_Data), and jumping to execute step S4.2) if failing to exceed the maximum line number of the regrouped data (Grouped_Data); otherwise, jumping to execute step S5).

The present invention further provides a gene sequencing quality line data compression system, including a computer system, wherein computer equipment is programmed to execute the steps of gene sequencing quality line data compression pre-processing method provided by the present invention.

The present invention further provides a gene sequencing quality line data compression system, including a computer system, wherein the computer equipment is executed to execute the steps of gene sequencing quality line data decompression and restoration method provided by the present invention.

The gene sequencing quality line data compression pre-processing method has the following technical effects:

1. The quality lines with the same gene sequencing result are gathered to improve the compression efficiency. Through the analysis for gene sequencing data, it is found that the quality lines are similar, all of which have the strong similarity on some columns, especially the detection results of the first several columns are importantly associated with the detection quality of the entire quality line, and these columns can be used as the index columns. According to the present invention, the quality lines having the same index column are gathered to get the quality line data having the similar gene detection qualities together, so that the subsequent compression algorithm is good in compression effect.

2. The bigger the data block input, the better the effect. For the method provided by the present invention, the bigger the data block to be pressed, the more the quality lines having the same index column information, the more the quality line data gathered in the same group, so that the better compression ration can be obtained by the subsequent compression.

3. There is no extra storage overhead in the compression result. The result of the method provided by the present invention after compression pre-processing includes: (Grouped_Data), (Index_Data) and (Index_No), wherein the (Index_Data) is index column information extracted from the original data block, and the (Grouped_Data) is other data with the index column information removed after the quality lines are re-organized. (Index_No) is index column number information. Generally, there is a few of index columns, and the index column numbers can be recorded by several bytes only. Under normal circumstances, a default value can be selected for the (Index_No.), without saving the (Index_No). Hence, the (Index_No) is not stored if the defaulted index column numbers are used directly in the method provided by the present invention, and no any extra storage overhead will be caused. If other index column acquisition methods are applied, the extra overhead for several bytes is only increased to save the index column numbers. The extra overhead can be ignored relative to the quality line data of several GBs.

4. Small computation overhead. Due to the fact that the calculation overhead for the compression pre-processing according to the method provided by the invention is small upon optimization, the quality line data of 4 GB can be processed for about 2 s to completely conform to the demand for processing the gene sequencing data in real time.

The gene sequencing quality line data decompression and restoration method provided by the present invention is a reverse method corresponding to the gene sequencing quality line data compression pre-processing method provided by the present invention, and has the corresponding advantages of the gene sequencing quality line data compression pre-processing method provided by the invention, so it will not be further explained herein. The gene sequencing quality line data compression system provided by the present invention is programmed to execute the steps of the gene sequencing quality line data compression pre-processing method or the gene sequencing quality line data decompression and restoration method provided by the present invention, and similarly has the corresponding advantages of the gene sequencing quality line data compression pre-processing method provided by the present invention, so it will not be further explained herein.

### BRIEF DESCRIPTION OF THE DRAWINGS

FIG. 1 is a basic flow diagram of a compression pre-processing method in the embodiments of the present invention.

FIG. 2 is a basic flow diagram of a decompression and restoration method in the embodiments of the present invention.

### DESCRIPTION OF THE EMBODIMENTS

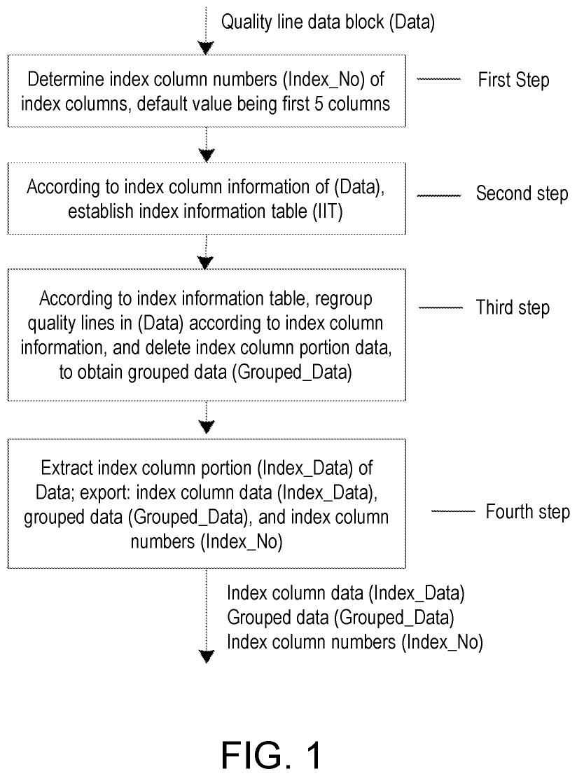

As shown in FIG. 1, the gene sequencing quality line data compression pre-processing method in this embodiment includes the following implementation steps:

1) reading an original data block (Data) of the quality line data and determining an index column numbers (Index_No) thereof;

2) establishing an index information table (IIT) according to the index columns of an original data block (Data);

3) according to the index information table (IIT), regrouping quality lines in the original data block (Data) according to the index column information, and deleting index column portion data to obtain grouped data (Grouped_Data);

4) extracting index column data (Index_Data) of the original data block (Data), and exporting the index column numbers (Index_No), index column data (Index_Data) of the original data block (Data) and data (Grouped_Data) regrouped as the compression pre-processing results.

In this embodiment, a function for the index column number (Index_No) in step 1) is determined as:

Get_Index_Column(Data)

by default, the function (Get_Index_Column) is directly returned to the first 5 columns of the quality line data as the index columns, that is, Index_No={0,1,2,3,4}. Besides, other columns or column numbers can be formulated according to the needs.

In this embodiment, step 2) includes the following detailed steps:

2.1) initializing the number of entries of the index information table (IIT) to be 0, and including serial numbers, index column information (Index), and variables (num, start and temp) in entries of the index information table (IIT) structurally, wherein the variable (num) is the number of quality lines having the corresponding index column information; the variable (start) indicates initial locations of the quality lines having the index column information after grouped; the variable (temp) is the number of quality lines having the corresponding index column information in regrouping.

2.2) initializing the current quality line number (i) of the original data block (Data) to be 0;

2.3) sequentially scanning the current quality line (Data[i]) in the original data block (Data), and jumping to execute step 2.6) if reaching the end of the original data block (Data); otherwise, taking out the index column information (Index) of the current quality line (Data[i]), wherein (Data[i]) refers to the contents of the current quality line (i) in the original data block (Data), namely Index=get_index(Data[i], Index_No); adding 1 to the current quality line number (i);

2.4) searching all entries in the index information table (IIT), adding 1 to the variable (num) of the entry (j) (IIT[j].num=IIT[j].num+1) if the index column information of a certain entry (j) of the index information table (IIT) is equal to the index column information (Index) of the current quality line (Data[i]) (IIT[j].Index=Index), and jumping to execute step 2.3); otherwise, skip to execute step 2.5);

2.5) establishing a new entry (k) in the index information table (IIT), setting index column information (IIT) ([k].Index) of an entry (k) to be equal to index column information (Index) of the current quality line (Data[i]) (IIT[k].Index=Index), and the variable (num) of the entry (k) to be equal to 1 (IIT[k].num=1), and adding 1 to a serial number (k) (k=k+1); jumping to execute step 2.3);

2.6) initializing the current entry (j) of the index information table (IIT) to be 0;

2.7) sequentially scanning the entries of the index information table (IIT), setting corresponding grouping start positions for all index column information, and ending this step and jumping to execute step 3) if reaching the end of the index information table (IIT); otherwise, with respect to the entry (j) scanned currently in the index information table (IIT), setting the value of the variable (start) of the entry (j) to be 0 and the value of variable (temp) to be 0 if the serial number of the entry (j) is 0, and adding 1 to (j), namely:

IIT[j].start=0; IIT[j].temp=0; j=j+1; jumping to continue with step 2.7);

otherwise setting the value of the variable (start) of the entry (j) to be the sum of the variables (start and num) of the last entry (j−1) and the variable (temp) of the entry (j) to be 0, adding 1 to (j), namely:

IIT[j].start=IIT[j−1].start+IIT[j−1].num; j=j+1; IIT[j].temp=0;jumping to continue with step 2.7).

In this embodiment, step 3) includes the following detailed steps:

3.1) allocating a space for the regrouped data (Grouped_Data), wherein the number of lines thereof is the same as that of the original data block (Data);

3.2) initializing the value of the current quality line number (i) of the original data block (Data) to be 0;

3.3) scanning the current quality line of the original data block (Data), wherein the data of the current quality line is Data[i], and (i) is the current quality line number; taking out the index column information (Index) of the current quality line Data[i];

3.4) searching the entry (j), the index information of which is the same as (Index), in the index information table (IIT) (namely in conformity with IIT[j].Index=Index);

3.5) inserting the quality line data, the index column information of which is deleted, into the regrouped data (Grouped_Data) (Grouped_Data[k]=delete index(Data[i], Index_No)), wherein a value of an insertion position (k) is the sum of the variables (start and temp) of the entry (j) (k=IIT[j].start+IIT[j].temp); adding 1 to the variable (temp) value of the entry (j) (IIT [j].temp=IIT [j].temp+1);

3.6) adding 1 to the line number (i) (i=i+1), judging whether the line number (i) is more than the total line number of the original data block (Data), and jumping to execute step 3.3) if the total line number of the original data block (Data) is not exceeded; otherwise, jumping to execute step 4).

In this embodiment, when the index column data (Index_Data) of the original data block (Data) is extracted in step 4), taking out the index columns of all quality lines from the original data block (Data) in an order from small to large according to the index column numbers (Index_No), so as to obtain the index column data (Index_Data), namely Index_Data=get_index_all(Data, Index_No); and finally, exporting the index column numbers (Index_No), the index column data (Index_Data) of the original data block (Data) and data (Grouped_Data) regrouped as the compression pre-processing results.

The gene sequencing quality line data compression pre-processing method in this embodiment puts forward a Grouped by Index Columns (GIC) based compression pre-processing method, wherein the basic idea thereof is to extract several columns from an inputted quality line document or data block to act as index columns, and then perform rearrangement on all quality line data, all quality lines having a same index column being one group and being arranged together according to their relative positions in the original data block. Since quality line data having a same index column is usually more similar, the data reorganization means can arrange similar quality line data together in the gene sequencing result, so as to increase local similarity of the data. The compression efficiency of the gene sequencing data can be further improved by performing BWT conversion and subsequent compression for the data subject to the GIC based compression pre-processing method in this embodiment. The present invention does not introduce additional storage overhead, and uses only small computational overhead to implement data rearrangement within large data windows, so as to improve compression efficiency. The gene sequencing quality line data compression pre-processing method in this embodiment is suitable for performing compression pre-processing on quality line data in a gene sequencing result document (FASTQ), wherein the bigger the data block, the more significant the advantage. In this embodiment, the quality line data obtained by gene sequencing is input by the compression pre-processing portion of the gene sequencing quality line data compression pre-processing method. The volume of quality line data composed of many quality lines is high, generally hundreds of MBs every minute. According to the GIC based compression pre-processing method in this embodiment, the quality lines are rearranged based on each quality line information in the index columns to obtain the converted quality line data through the determination for the index columns. The quality line data, converted by the GIC based compression pre-processing method in this embodiment, is subject to the subsequent compression processing. With respect to the gene sequencing quality line data, the local similarity of the data can be improved by the gene sequencing quality line data compression pre-processing method in this embodiment in the large data block range, thereby improving the gene sequencing data compression efficiency.

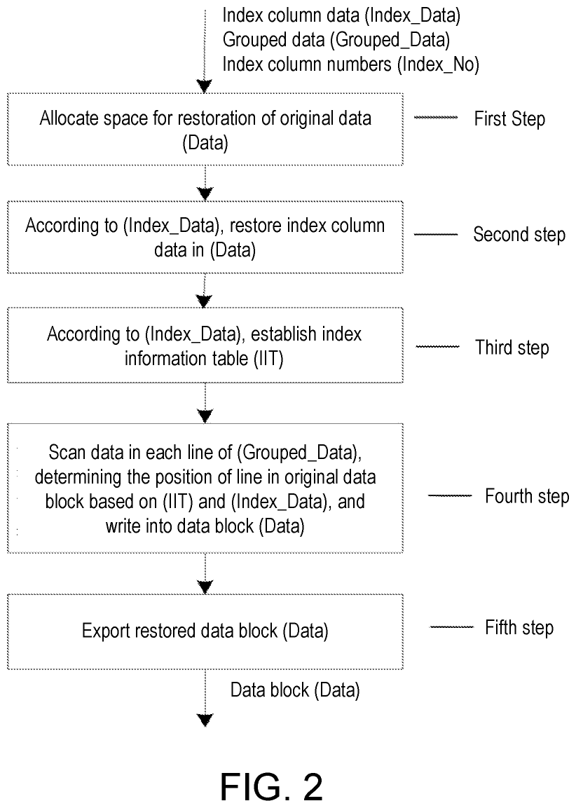

The decompression portion provided by the present invention is required to restore the original data block (Data) based on the index column data (Index_Data), the regrouped data (Grouped_Data) and the index column numbers (Index_No). Since the contents of the index column data (Index_Data) are the index column contents in the original data block (Data), it is easy to obtain the index information table according to the index column data (Index_Data). Then, the contents in the regrouped data (Grouped_Data) can be restored to the corresponding lines thereof in the original data block (Data) by the index information table, and then can be combined with the index column data (Index_Data), namely the original data block (Data) is restored. As shown in FIG. 2, the gene sequencing quality line data decompression and restoration method in this embodiment includes the following implementation steps:

S1) reading decompressed index column data (Index_Data), regrouped data (Grouped_Data) and index column numbers (Index_No), determining the quality line number of the original data block (Data) and character data of each line based on the regrouped data (Grouped_Data) and index column number information (Index_No), and allocating the space for the storage of the original data block (Data);

S2) according to the index column numbers (Index_No), respectively assigning each column of data among the index column data (Index_Data) to the corresponding column, the number of which belongs to Index_No, in the original data block (Data);

S3) establishing the index information table (IIT) according to the index column data (Index_Data);

S4) sequentially scanning each line of data among the regrouped data (Grouped_Data) according to the index information table (IIT), determining the position of the line in the original data block according to the index information table (IIT) and the index column data (Index_Data), and writing the same into the corresponding quality line of the original data block (Data);

S5) exporting the original data block (Data).

In this embodiment, step S3) includes the following detailed steps:

S3.1) initializing the value of the entry number (k) of the index information table (IIT) to be 0, and including serial numbers, index column information (Index), and variables (num, start and temp) in the entries of the index information table (IIT) structurally, wherein the variable (num) is the number of quality lines having the corresponding index column information; the variable (start) indicates the initial locations of the quality lines having the index column information after grouping; the variable (temp) is the number of quality lines having the corresponding index column information in data restoration;

S3.2) initializing the value of the current line number (i) of the index column data (Index_Data) to be 0;

S3.3) sequentially scanning the index column data (Index_Data), and jumping to execute step S3.6) if reaching the end of the index column data (Index_Data); otherwise, taking out the current index column information (Index_Data[i]) corresponding to the current line in the index column data (Index_Data);

S3.4) searching all entries in the index information table (IIT), adding 1 to the variable (num) of the entry (j) (IIT[j].num=IIT[j].num+1) if the index column information (Index) of the entry (j) is the same as the current index column information (Index_Data[i]), and jumping to execute step S3.3); otherwise, jumping to execute step 3.5);

S3.5) establishing a new entry (k) for the index information table (IIT), wherein the index column information (Index) of the entry (k) is equal to the current index column information (Index_Data[i]) (IIT[k].index=Index_Data[i]), and the variable (num) is equal to 1 (IIT[k].num=1); adding 1 to the entry number (k) (k=k+1), and jumping to execute step S3.3);

S3.6) initializing the current entry (j) of the index information table (IIT) to be 0;

S3.7) sequentially scanning the index information table (IIT), and setting the corresponding grouping start position of the current index column information; in case of reaching the end of the index information table (IIT), jumping to step S4); otherwise, with respect to the entry (j) in the index information table (IIT): if the serial number (j) of the entry (j) is 0, setting the variables (start and temp) to be 0, and adding 1 to the serial number (j), namely:

IIT[j].start=0; IIT[j].temp=0; j=j+1; jumping to continue with step S3.7);

otherwise setting the value of the variable (start) of the entry (j) to be the sum of the variables (start and num) of the last entry (j−1), adding 1 to the serial number (j), and setting the variable (temp) of the entry (j) to be 0, namely:

IIT[j].start=IIT[j−1].start+IIT[j−1].num; IIT[j].temp=0; j=j+1; jumping to continue with step S3.7);

In this embodiment, step S4) includes the following detailed steps:

S4.1) initializing the value of the current line number (k) of the regrouped data (Grouped_Data) to be 0;

S4.2) obtaining the index column information of the regrouped data (Grouped_Data[k]): if reaching the end of regrouped data (Grouped_Data), jumping to execute step S5); otherwise, scanning the index information table (IIT) to find out the entry (j) of the index information table (IIT) to make it conform to that: the value of the line number (k) is more than or equal to the value of the variable (start) of the entry (j), and less than or equal to the sum of the values of the variable (start) of the entry (j) and the variable (num) thereof (IIT[j].start≤k≤IIT[j].start+IIT[j].num), wherein the index column information corresponding to the data (Grouped_Data[k]) of the current line in the regrouped data (Grouped_Data) is the index column information (Index) (IIT[j].index) of the entry (j);

S4.3) combining the data (Grouped_Data[k]) of the current line in the regrouped data (Grouped_Data) and the index column information (Index) of the entry (j) (IIT[j].index) to generate a complete quality line (Temp_Read);

S4.4) obtaining an occurrence order (r) in the quality line having the same index column information of the complete quality line (Temp_Read) in the original data block (Data), wherein the value of the occurrence order (r) is a differential value between the current line number (k) and the variable (start) of the entry (j) (namely: r=k-IIT[j].start);

S4.5) sequentially scanning the index column data (Index_Data) to find out the r^{th} index column information to be an entry (t) of the index column information (Index) of the entry (j) in the index information table (IIT) (IIT[j].index), so as to determine the line number (t) of the complete quality line (Temp_Read) in the original data block;

S4.6) writing the complete quality line (Temp_Read) to the line number (t) of the original data block (Data) (Data[t]=Temp_Read);

S4.7) adding 1 to the current line number (k) of the regrouped data (Grouped_Data) (k=k+1);

S4.8) judging whether the current line number (k) is more than the maximum line number of the regrouped data (Grouped_Data), and jumping to execute step S4.2) if failing to exceed the maximum line number of the regrouped data (Grouped_Data); otherwise, jumping to execute step S5).

This embodiment further provides a gene sequencing quality line data compression system, including a computer system, wherein the computer equipment is programmed to execute the steps of the aforesaid gene sequencing quality line data compression pre-processing method in this embodiment.

This embodiment further provides a gene sequencing quality line data compression system, including a computer system, wherein the computer equipment is programmed to execute the steps of the aforesaid gene sequencing quality line data decompression and restoration method in this embodiment.

The above are only preferred embodiments of the present invention, and the protection scope of the present invention is not limited to the embodiments mentioned above. The technical solutions under the ideas of the present invention fall into the protection scope of the present invention. It should be pointed out that, for those of ordinary skill in the art, some improvements and modifications without departing from the principle of the present invention shall be deemed as the protection scope of the present invention.

1. A method of gene sequencing quality line data compression pre-processing, wherein the implementation steps comprise:
1) reading an original data block (Data) of the quality line data and determining an index column numbers (Index_No) thereof;
2) establishing an index information table (IIT) according to an index columns of the original data block (Data);
3) according to the index information table (IIT), regrouping quality lines in the original data block (Data) according to an index column information, and deleting portion of an index column data to obtain a regrouped data (Grouped_Data);
4) extracting the index column data (Index_Data) of the original data block (Data), and exporting the index column numbers (Index_No), the index column data (Index_Data) of the original data block (Data) and the regrouped data (Grouped_Data) as compression pre-processing results.

2. The method of gene sequencing quality line data compression pre-processing of claim 1, wherein step 2) comprises the following detailed steps:
2.1) initializing number of entries of the index information table (ITT) to be 0, and including serial numbers, the index column information (Index), and variables (num, start and temp) in entries of the index information table (IIT) structurally, wherein the variable (num) is a number of quality lines having the corresponding index column information; the variable (start) indicates initial locations of the quality lines having the index column information after grouped; the variable (temp) is the number of quality lines having the corresponding index column information in regrouping;
2.2) initializing a current quality line number (i) of the original data block (Data) to be 0;
2.3) sequentially scanning a current quality line (Data[i]) in the original data block (Data), and jumping to execute step 2.6) if reaching the end of the original data block(Data); otherwise, taking out the index column information (Index) of the current quality line (Data[i]), wherein (Data[i]) refers to contents of the current quality line (i) in the original data block (Data); adding 1 to the current quality line number (i);
2.4) searching all entries in the index information table (IIT), adding 1 to the variable (num) of an entry (j) if the index column information of a certain entry (j) of the index information table (IIT) is equal to the index column information (Index) of the current quality line (Data[i]), jumping to execute step 2.3); otherwise, jumping to execute step 2.5);
2.5) establishing a new entry (k) in the index information table (ITT), setting index column information (IIT[k].Index) of an entry (k) to be equal to the index column information (Index) of the current quality line (Data[i]), and the variable (num) of the entry (k) to be equal to 1, and adding 1 to a serial number (k); jumping to execute step 2.3);
2.6) initializing the current entry (j) of the index information table (ITT) to be 0;
2.7) sequentially scanning the entries of the index information table (IIT), setting corresponding grouping start positions for all index column information, and ending this step and jumping to execute step 3) if reaching the end of the index information table (IIT); otherwise, with respect to the entry (j) scanned currently in the index information table (IIT), setting a value of the variable (start) of the entry (j) to be 0 and a value of variable (temp) to be 0 if a serial number (j) of the entry is 0, and adding 1 to the serial number (j) of the current entry; jumping to continue with step 2.7); otherwise setting the value of the variable (start) of the entry (j) to be the sum of the variable (start) and the variable (num) of the last entry (j−1) and the value of the variable (temp) of the entry (j) to be 0, adding 1 to the serial number (j) of the current entry, and jumping to continue with step 2.7).

3. The method of gene sequencing quality line data compression pre-processing of claim 1, wherein step 3) comprises the following detailed steps:
3.1) allocating a space for the regrouped data (Grouped_Data), wherein a number of lines thereof is the same as that of the original data block (Data);
3.2) initializing a value of a current quality line number (i) of the original data block (Data) to be 0;
3.3) scanning a current quality line of the original data block (Data), wherein a current quality line data is Data[i], and (i) is the current quality line number; taking out the index column information (Index) of the current quality line Data[i];
3.4) searching an entry (j), an index information of which is the same as Index, in the index information table (IIT);
3.5) inserting the quality line data, the index column information of which is deleted, into the regrouped data (Grouped_Data), wherein a value of an insertion position (k) is the sum of the variables (start and temp) of the entry (j); adding 1 to a value of the variable (temp) of the entry (j);
3.6) adding 1 to the line number (i), judging whether the line number (i) is more than a total line number of the original data block (Data), and jumping to execute step 3.3) if the total line number of the original data block (Data) is not exceeded; otherwise, jumping to execute step 4).

4. A method of gene sequencing quality line data decompression and restoration, wherein the implementation steps comprise:
S1) reading decompressed index column data (Index_Data), regrouped data (Grouped_Data) and index column numbers (Index_No), determining a number of the quality line of an original data block (Data) and character data of each line based on the regrouped data (Grouped_Data) and index column number (Index_No), and allocating a space for storage of the original data block (Data);
S2) according to the index column numbers (Index_No), respectively assigning each column of data among the index column data (Index_Data) to the corresponding column, the number of which is recorded by Index_No, in the original data block (Data);
S3) establishing an index information table (IIT) according to the index column data (Index_Data)
S4) sequentially scanning each line of data among the regrouped data (Grouped_Data) according to the index information table (IIT), determining a position of the line in the original data block according to the index information table (ITT) and the index column data (Index_Data), and writing the same into the corresponding quality line of the original data block (Data);
S5) exporting the original data block (Data).

5. The method of gene sequencing quality line data decompression and restoration of claim 4, wherein step S3) comprises the following detailed steps:
S3.1) initializing a value of an entry number (k) of the index information table (IIT) to be 0, and including serial numbers, index column information (Index), and variables (num, start and temp) in entries of the index information table (ITT) structurally, wherein the variable (num) is a number of quality lines having the corresponding index column information; the variable (start) indicates initial locations of the quality lines having the index column information after grouping; the variable (temp) is the number of quality lines having the corresponding index column information in data restoration;
S3.2) initializing a value of a current line number (i) of the index column data (Index_Data) to be 0;
S3.3) sequentially scanning the index column data (Index_Data), and jumping to execute step S3.6) if reaching the end of the index column data (Index_Data); otherwise, taking out a current index column information (Index_Data[i]) corresponding to the current line in the index column data (Index_Data);
S3.4) searching all entries in the index information table (ITT), adding 1 to the variable (num) of an entry (j) if the index column information (Index) of the entry (j) is the same as the current index column information (Index_Data[i]), and jumping to execute step S3.3); otherwise, jumping to execute step 3.5);
S3.5) establishing a new entry (k) for the index information table (IIT), wherein the index column information (Index) of the entry (k) is equal to the current index column information (Index_Data[i]), and the variable (num) is equal to 1; adding 1 to the entry number (k), and jumping to execute step S3.3);
S3.6) initializing a current entry (j) of the index information table (ITT) to be 0;
S3.7) sequentially scanning the index information table (IIT), and setting corresponding grouping start position for the current index column information; if reaching the end of the index information table (IIT), jumping to step S4); otherwise, with respect to the entry (j) in the index information table (IIT): if a serial number (j) of the entry (j) is 0, setting the variables (start and temp) to be 0, adding 1 to the serial number (j), and jumping to continue with step S3.7); otherwise, setting the variable (start) of the entry (j) to be the sum of the variables (start) and the variables (num) of the last entry (j−1), wherein the variable (temp) of the entry (j) is 0, adding 1 to the serial number (j), and jumping to continue with step S3.7).

6. The method of gene sequencing quality line data decompression and restoration of claim 4, wherein step S4) comprises the following detailed steps:
S4.1) initializing a value of a current line number (k) of the regrouped data (Grouped_Data) to be 0;
S4.2) obtaining the index column information of the regrouped data (Grouped_Data[k]): if reaching the end of regrouped data (Grouped_Data), jumping to execute step S5); otherwise, scanning the index information table (IIT) to find out an entry (j) of the index information table (IIT) to make it conform to that: a value of a line number (k) is more than or equal to a value of the variable (start) of the entry (j), and less than or equal to the sum of values of the variable (start) of the entry (j) and the variable (num) thereof, wherein the index column information corresponding to data (Grouped_Data[k]) of the current line in the regrouped data (Grouped_Data) is the index column information (Index) of the entry (j);
S4.3) combining the data (Grouped_Data[k]) of the current line in the regrouped data (Grouped_Data) and the index column information (Index) of the entry (j) to generate a complete quality line (Temp_Read);
S4.4) obtaining an occurrence order (r) in the quality line having the same index column information of the complete quality line (Temp_Read) in the original data block (Data), wherein a value of the occurrence order (r) is a differential value between the current line number (k) and the variable (start) of the entry (j);
S4.5) sequentially scanning the index column data (Index_Data) to find out the r^{th} index column information to be an entry (t) of the index column information (Index) of the entry (j) in the index information table (IIT), so as to determine a line number (t) of the complete quality line (Temp_Read) in the original data block;
S4.6) writing the complete quality line (Temp_Read) to the line number (t) of the original data block (Data);
S4.7) adding 1 to the current line number (k) of the regrouped data (Grouped_Data);
S4.8) judging whether the current line number (k) is more than the maximum line number of the regrouped data (Grouped_Data), and jumping to execute step S4.2) if failing to exceed the maximum line number of the regrouped data (Grouped_Data); otherwise, jumping to execute step S5).

7. A gene sequencing quality line data compression system, comprising a computer system, wherein the computer equipment is programmed to execute the steps of the method of gene sequencing quality line data compression pre-processing of claim 1.

8. A gene sequencing quality line data compression system, comprising a computer system, wherein the computer equipment is programmed to execute the steps of the method of gene sequencing quality line data decompression and restoration of claim 4.

9. A gene sequencing quality line data compression system, comprising a computer system, wherein the computer equipment is programmed to execute the steps of the method of gene sequencing quality line data compression pre-processing of claim 2.

10. A gene sequencing quality line data compression system, comprising a computer system, wherein the computer equipment is programmed to execute the steps of the method of gene sequencing quality line data compression pre-processing of claim 3.

11. A gene sequencing quality line data compression system, comprising a computer system, wherein the computer equipment is programmed to execute the steps of the method of gene sequencing quality line data decompression and restoration of any of claim 5.

12. A gene sequencing quality line data compression system, comprising a computer system, wherein the computer equipment is programmed to execute the steps of the method of gene sequencing quality line data decompression and restoration of any of claim 6.
